(19) United States
(12) Patent Application Publication
(10) Pub. No.: US 2023/0157558 A1
(43) Pub. Date: May 25, 2023

(54) CUFF FOR MEASURING BLOOD PRESSURE

(71) Applicant: OMRON HEALTHCARE Co., Ltd., Kyoto (JP)

(72) Inventors: Ryosuke DOI, Kyoto (JP); Minoru TANIGUCHI, Kyoto (JP); Takayuki MATSUOKA, Kyoto (JP); Takayuki SHIINA, Kyoto (JP); Yo IMAI, Kyoto (JP)

(21) Appl. No.: 18/157,609

(22) Filed: Jan. 20, 2023

Related U.S. Application Data
(63) Continuation of application No. PCT/JP2021/025669, filed on Jul. 7, 2021.

(30) Foreign Application Priority Data
Jul. 27, 2020 (JP) 2020-126734

(51) Int. Cl. A61B 5/022

(52) U.S. Cl. CPC A61B 5/02233

(57) ABSTRACT

To provide a cuff for measuring blood pressure which is excellent in durability and convenient to use. A cuff for measuring blood pressure includes a band enveloping an air bag between an outer cloth and an inner cloth facing the outer cloth; a first opening portion provided in the outer cloth; and a first hook-and-loop fastener welded to the outer cloth in a state where the first opening portion is closed from an inner side of the band and a raised surface is exposed through the first opening portion.

### CROSS-REFERENCE TO RELATED APPLICATIONS

This application is the U.S. national stage application filed pursuant to 35 U.S.C. 365(c) and 120 as a continuation of International Patent Application No. PCT/JP2021/025669, filed Jul. 7, 2021, which application claims priority to Japanese Patent Application No. 2020-126734, filed Jul. 27, 2020, which applications are incorporated herein by reference in their entireties.

### TECHNICAL FIELD

The present invention relates to a cuff for measuring blood pressure used in a sphygmomanometer for measuring blood pressure.

### BACKGROUND ART

JP 06031859 B discloses a folding-back type compression band for a blood pressure measurement device. The compression band for the blood pressure measurement device has a configuration in which a hook-and-loop fastener is sewn on a front surface of an outer bag that stores an air bag. The outer bag of the compression band for the blood pressure measurement device is formed by welding an outer member and an inner member. At one end of the compression band for the blood pressure measurement device, a ring member through which the other end of the compression band for the blood pressure measurement device is passed and folded back is provided. A portion of the ring member is held between the outer member and the inner member. Further, the ring member is supported by the outer bag by folding back and sewing the one end of the compression band for the blood pressure measurement device to the inner member side.

JP 06098237 B describes a cuff for measuring blood pressure. This cuff for measuring blood pressure includes a bag-shaped band formed by welding a raised outer cloth and an inner cloth. The inner cloth is provided with a hook-and-loop fastener. A ring member for passing one end of the band therethrough is attached to the outer cloth by a ring attachment member. The ring attachment member is formed by folding back the same cloth material as the outer cloth so as to surround one side of the ring member and sewing the folded back portion on the remaining flat portion. A visible portion on the front side of the ring attachment member is raised to be engageable with the hook-and-loop fastener. A configuration is disclosed in which a flat portion of the ring attachment member is welded to the outer cloth.

### SUMMARY OF INVENTION

### Technical Problem

In a configuration in which the hook-and-loop fastener is fixed by sewing to a portion of the band that is passed through the ring member as in JP 06031859 B, when the portion of the hook-and-loop fastener in the band is passed through the ring member, there is a possibility that an outer edge portion of the sewn portion of the hook-and-loop fastener be turned up. Frequent occurrence of the turning-up leads to peeling off of the hook-and-loop fastener. In addition, when the turning-up occurs, there is a problem that usability of the compression band is adversely affected, for example, workability when the compression band is wound around an arm is lowered.

How the hook-and-loop fastener disclosed in JP 06098237 B is fixed is not particularly described and is unclear. However, in a configuration in which the hook-and-loop fastener is fixed by sewing, there is concern that the hook-and-loop fastener may be turned up similarly.

The turning-up of the hook-and-loop fastener may occur not only when the hook-and-loop fastener is passed through the ring member but also, for example, when a user touches the hook-and-loop fastener with his/her hand or when the hook-and-loop fastener is repeatedly engaged with and attached or detached from a raised surface.

The purpose of the present invention is to provide a cuff for measuring blood pressure which is excellent in durability and convenient to use.

### Solution to Problem

(1): A cuff for measuring blood pressure including a band enveloping a fluid bag between a first skin member and a second skin member facing the first skin member; a first opening portion provided in the first skin member; and a first hook-and-loop fastener welded to the first skin member in a state where the first opening portion is closed from an inner side of the band and a fastener surface is exposed through the first opening portion.

According to (1), since the first hook-and-loop fastener is provided on the inner side of the band, it is possible to prevent an object from being caught by the first hook-and-loop fastener when the band is worn, for example. As a result, the first hook-and-loop fastener is prevented from being peeled off, so that the durability of the cuff for measuring blood pressure can be improved. In addition, since the first hook-and-loop fastener is attached from the inner side of the first opening portion, the fixed portion is not exposed to the outer side of the first skin member. Therefore, when the band is wound around an arm, it is possible to prevent the band from being caught, improving workability. The first hook-and-loop fastener is welded to the first skin member. Therefore, the cuff for measuring blood pressure can be easily manufactured and the manufacturing cost can be reduced as compared with the conventional sewing. Further, as compared with a configuration in which the first hook-and-loop fastener is fixed to the outer surface of the skin member by sewing, the appearance quality of the cuff for measuring blood pressure can be improved because the sewn portion and the fastener end edges are not visible.

(2): The cuff for measuring blood pressure according to (1), wherein the first skin member includes a first member and a second member formed on an inner side of the first member and containing a resin, and an opening edge portion of the first opening portion in the second member is welded to an edge side portion of the fastener surface of the first hook-and-loop fastener.

According to (2), the first hook-and-loop fastener and the first skin member can be welded to each other by utilizing the characteristics of a material generally used as the first skin member. Therefore, an existing raw material of the cuff for measuring blood pressure can be used as it is, and an increase in manufacturing cost can be prevented.

(3): The cuff for measuring blood pressure according to (1) or (2), further including a second opening portion provided in the first skin member; and a second hook-and-loop fastener welded to the first skin member in a state where the second opening portion is closed from the inner side of the band and a fastener surface is exposed through the second opening portion, wherein the first hook-and-loop fastener and the second hook-and-loop fastener are engageable with each other.

According to (3), each of the first hook-and-loop fastener and the second hook-and-loop fastener is provided on the inner side of the band. Thus, it is possible to prevent an object from being caught by the first hook-and-loop fastener and the second hook-and-loop fastener when the band is worn, for example. In addition, since the first hook-and-loop fastener and the second hook-and-loop fastener are not attached from the outer surface side of the band, the appearance quality can be improved.

(4): The cuff for measuring blood pressure according to (3), wherein the first skin member includes a first member and a second member formed on the inner side of the first member and containing a resin, the first skin member constitutes a surface of the band on a side opposite to a side to be in contact with a target measurement site, and a fastener rear surface member having stretchability equal to or lower than stretchability of the first member is fixed to a surface opposite to the fastener surface of at least the first hook-and-loop fastener of the first hook-and-loop fastener and the second hook-and-loop fastener.

According to (4), in the first hook-and-loop fastener, the fastener rear surface member having the stretchability equal to or lower than the stretchability of the first member is provided on the rear surface side of the surface exposed through the first opening portion. Therefore, an increase in the stretchability of the first skin member due to the provision of the first opening portion can be suppressed by the fastener rear surface member. As a result, it is possible to realize the compression state of the target measurement site suitable for blood pressure measurement.

(5): The cuff for measuring blood pressure according to (4), wherein an area of the fastener surface of the first hook-and-loop fastener exposed through the first opening portion is larger than an area of the fastener surface of the second hook-and-loop fastener exposed through the second opening portion.

According to (5), the fastener rear surface member is provided on the rear surface side of the first hook-and-loop fastener occupying a large proportion in the first skin member. Therefore, the stretchability of the first skin member as a whole can be sufficiently and appropriately adjusted. As a result, it is possible to compress an arm suitably for blood pressure measurement.

(6): The cuff for measuring blood pressure according to (4), wherein the first hook-and-loop fastener is disposed closer to the fluid bag than the second hook-and-loop fastener is.

According to (6), the fastener rear surface member is provided on the rear surface side of the first hook-and-loop fastener on the side near the fluid bag which greatly contributes to the compression of the target measurement site. Therefore, it is possible to adjust the stretchability of the first skin member in the vicinity of the fluid bag so as to be suitable therefor. As a result, it is possible to compress an arm suitably for blood pressure measurement.

(7): The cuff for measuring blood pressure according to any one of (4) to (6), wherein the fastener rear surface member is fixed only to the first hook-and-loop fastener of the first hook-and-loop fastener and the second hook-and-loop fastener.

According to (7), since the second hook-and-loop fastener is not provided with the fastener rear surface member, it is possible to simplify the configuration and reduce the manufacturing cost.

### Advantageous Effects of Invention

According to the present invention, it is possible to provide a cuff for measuring blood pressure which is excellent in durability and convenient to use.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a plan view of a cuff for measuring blood pressure according to an embodiment of the present invention when viewed from a direction of an outer surface;

FIG. 2 is a plan view of the cuff for measuring blood pressure illustrated in FIG. 1 when viewed from a direction of an inner surface;

FIG. 3 is an exploded perspective view of the cuff for measuring blood pressure illustrated in FIG. 1;

FIG. 4 is a cross-sectional view of a portion taken along line A-A of FIG. 1;

FIG. 5 is an enlarged cross-sectional view of an outer cloth and an inner cloth of the cuff for measuring blood pressure illustrated in FIG. 1;

FIG. 6 is an enlarged schematic cross-sectional view illustrating a state before a first hook-and-loop fastener is welded in manufacturing the cuff for measuring blood pressure illustrated in FIG. 1;

FIG. 7 is an enlarged schematic cross-sectional view illustrating a welded state of the first hook-and-loop fastener illustrated in FIG. 1;

FIG. 8 is an enlarged perspective view illustrating an end portion on a side where a second hook-and-loop fastener is provided in the cuff for measuring blood pressure illustrated in FIG. 1;

FIG. 9 is a cross-sectional view of a portion taken along line B-B of FIG. 8;

FIG. 10 is an enlarged perspective view illustrating a terminal region of the cuff for measuring blood pressure illustrated in FIG. 1;

FIG. 11 is an enlarged cross-sectional view of a portion taken along line C-C of the terminal region illustrated in FIG. 10; and,

FIG. 12 is a schematic cross-sectional view illustrating a state where the cuff for measuring blood pressure illustrated in FIG. 1 is being worn on a target measurement site.

### DESCRIPTION OF EMBODIMENTS

### Overview of Cuff for Measuring Blood Pressure of Embodiment

First, an outline of an embodiment of a cuff for measuring blood pressure of the present invention will be described. A cuff for measuring blood pressure of the embodiment has a structure in which a hook-and-loop fastener of a band used when the band is wound around and fixed to a target measurement site such as an upper arm part is welded from an inner side of a skin member of the band so as to close an opening portion formed in the skin member. With such a structure, an outer edge portion of the hook-and-loop fastener is not exposed so that an object is prevented from being caught.

Hereinafter, a specific configuration example of the cuff for measuring blood pressure of the embodiment will be described. Various methods such as thermal welding, high-frequency welding, and ultrasonic welding can be employed as the welding method of the two members described below.

### EMBODIMENT

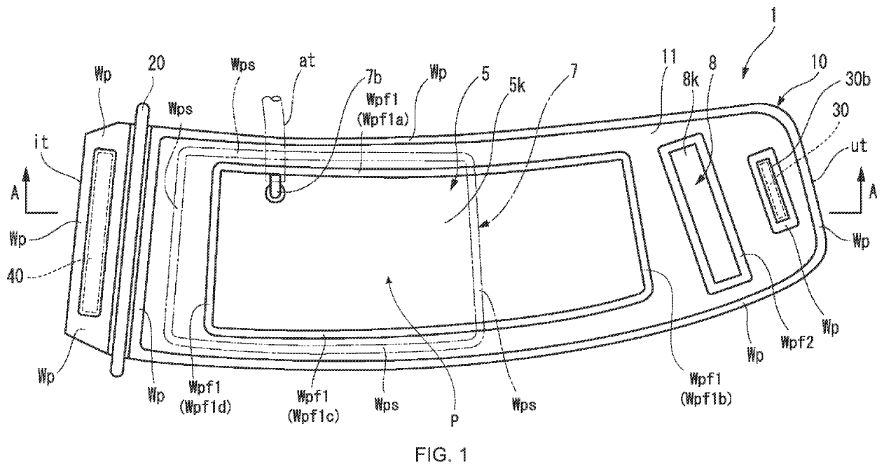

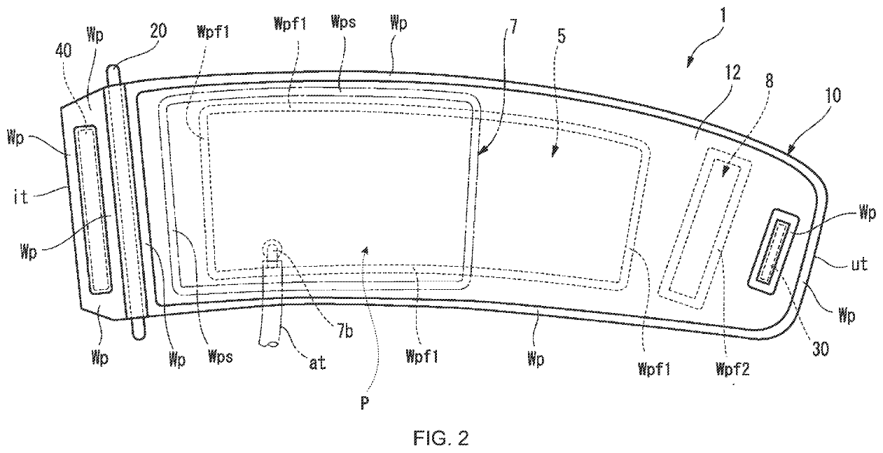

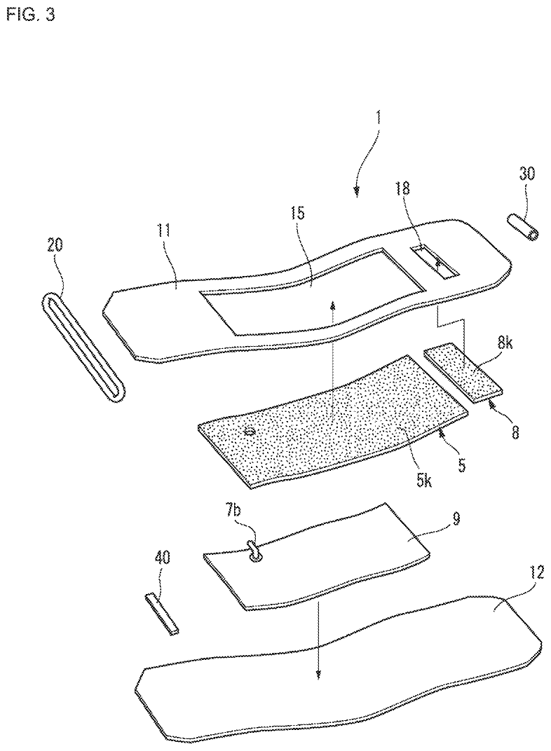

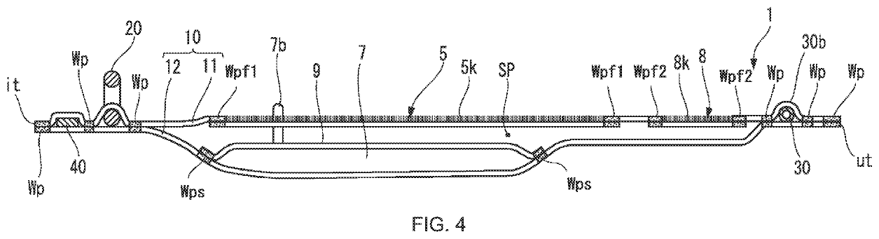

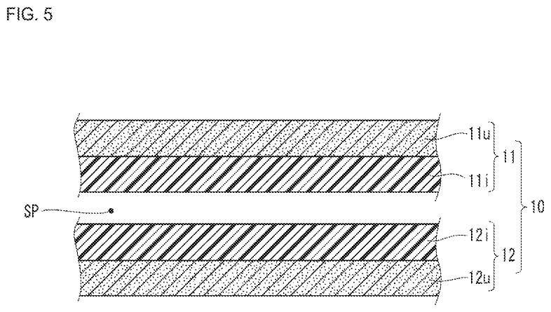

FIG. 1 is a plan view of a cuff for measuring blood pressure 1 according to an embodiment of the present invention when viewed from a direction of an outer surface. FIG. 2 is a plan view of the cuff for measuring blood pressure 1 illustrated in FIG. 1 when viewed from a direction of an inner surface. FIG. 3 is an exploded perspective view of the cuff for measuring blood pressure 1 illustrated in FIG. 1. FIG. 4 is a cross-sectional view of a portion taken along line A-A of FIG. 1. FIG. 5 is an enlarged cross-sectional view of an outer cloth 11 and an inner cloth 12 of the cuff for measuring blood pressure 1 illustrated in FIG. 1.

As illustrated in FIGS. 1 and 2, the cuff for measuring blood pressure 1 includes a band 10 including an outer cloth 11 constituting a first skin member and an inner cloth 12 constituting a second skin member and facing the outer cloth 11. The outer cloth 11 and the inner cloth 12 are partly fixed to each other to form a storage space SP (see FIG. 4) therebetween. An air bag 7 constituting a fluid bag is stored in the storage space SP. The fluid bag is a bag that can be supplied with and discharge a fluid such as air and is inflated by the supply of the fluid. The air bag 7 can be supplied with and discharge air as a fluid.

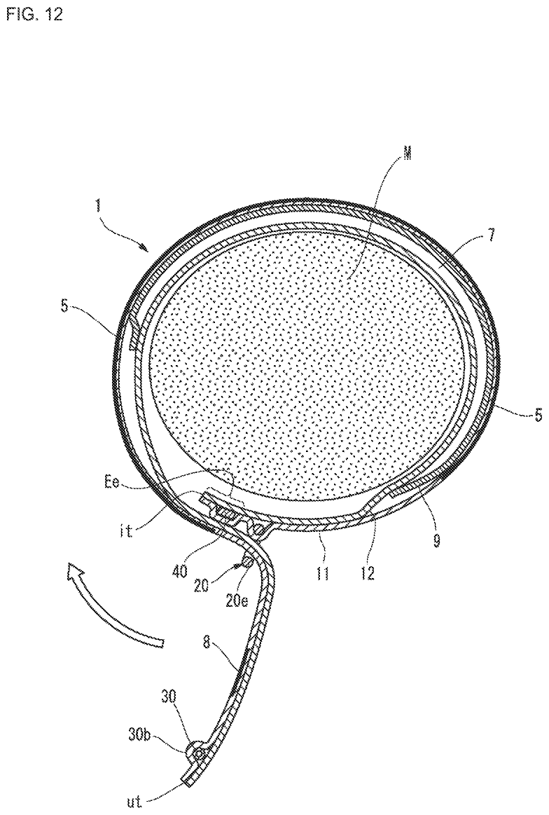

The band 10 has a band shape extending in a circumferential direction of an upper arm part M, which is a target measurement site, in a state of being wound around the upper arm part M (see FIG. 12). The circumferential direction in this state is a longitudinal direction of the band 10, and an axial direction of the upper arm part M is a lateral direction (width direction) of the band 10. Hereinafter, the longitudinal direction of the band 10 is also simply referred to as a longitudinal direction, and the lateral direction of the band 10 is also simply referred to as a lateral direction.

The inner cloth 12 constitutes a surface on a side in contact with the upper arm part M in a state where the band 10 is worn on the upper arm part M illustrated in FIG. 12. The outer cloth 11 constitutes a surface opposite to the upper arm part M side in the state where the band 10 is worn on the upper arm part M illustrated in FIG. 12. In the present embodiment, one end edge of the band 10 in the longitudinal direction is referred to as an end edge it, and the other end edge of the band 10 in the longitudinal direction is referred to as an end edge ut. The outer cloth 11 and the inner cloth 12 are joined together at their peripheries by welding as will be described later to form an exterior bag body, and the air bag 7 is located therein. In the present embodiment, a region where the outer cloth 11 and the inner cloth 12 are welded to each other is indicated by a welded region Wp. The welded region Wp constitutes a first welded region.

As illustrated in FIG. 5, each of the outer cloth 11 and the inner cloth 12 has, for example, a multi-layer structure. The inner cloth 12 includes a first member 12u having a sheet-like shape and made of a polyester cloth, a nylon cloth, or the like knitted with high stretchability to compress the target measurement site, and a second member 12i having a sheet-like shape, made of a PVC (polyvinylchloride) film or the like, and bonded to the inner side (storage space SP side) of the first member 12u. The first member 12u constitutes a surface member. The second member 12i constitutes a rear surface member.

The outer cloth 11 includes a sheet-like first member 11u made of a polyester cloth, a nylon cloth, or the like knitted with extremely low stretchability (with substantially no stretchability) as compared with the inner cloth 12, and a sheet-like second member 11i made of a PVC film or the like and bonded to the inner side (storage space SP side) of the first member 11u. Each of the outer cloth 11 and the inner cloth 12 has a multi-layer structure, which is effective for adjusting the stretchability and strength of each of the outer cloth 11 and the inner cloth 12.

Each of the second member 11i of the outer cloth 11 and the second member 12i of the inner cloth 12 may be made of a material suitable for welding. Examples of such a material include a material containing a resin such as a thermoplastic resin. The welded region Wp described above is a region formed by welding the second member 11i and the second member 12i.

As illustrated in FIG. 3, the cuff for measuring blood pressure 1 has a configuration in which a first hook-and-loop fastener 5, a second hook-and-loop fastener 8 having a smaller planar area than the first hook-and-loop fastener 5, a bag sheet member 9 with which the air bag 7 is formed, an oblong ring-like member 20, a tubular stopper tube 30, and a plate-like auxiliary member 40 are incorporated between the outer cloth 11 and the inner cloth 12.

In the outer cloth 11, a first opening portion 15 which is a rectangular opening and a second opening portion 18 which is a rectangular opening smaller than the first opening portion 15 are formed. The shape of each of the first opening portion 15 and the second opening portion 18 is rectangular, but is not limited thereto, and any shape can be adopted. The second opening portion 18 is provided in the vicinity of the end edge ut of the band 10. The first opening portion 15 is provided between the second opening portion 18 and the end edge it of the band 10. As illustrated in FIG. 4, the first opening portion 15 and the second opening portion 18 are formed in a portion of the outer cloth 11 that forms the storage space SP.

The first opening portion 15 is correspondingly provided with the first hook-and-loop fastener 5. The first hook-and-loop fastener 5 is welded to the outer cloth 11 in a state where the first opening portion 15 is closed from the storage space SP side. In the present specification, a region of the outer cloth 11 to which the first hook-and-loop fastener 5 is welded is referred to as a welded region Wpf1. In the example of FIGS. 1 and 2, the welded region Wpf1 has a substantially rectangular frame shape. The welded region Wpf1 constitutes a third welded region. A portion of the welded region Wpf1 extending in the lateral direction at an end portion on the end edge ut side is referred to as a welded region Wpf1b. A portion of the welded region Wpf1 extending in the lateral direction at an end portion on the end edge it side is referred to as a welded region Wpf1d. Portions of the welded region Wpf1 extending in the longitudinal direction at both end portions in the lateral direction are referred to as welded regions Wpf1a and Wpf1c.

A raised surface 5k, which is a fastener surface, is formed on the surface of the first hook-and-loop fastener 5. The first hook-and-loop fastener 5 is welded to the outer cloth 11 from the inner side (storage space SP side) of the band 10, in a state where the raised surface 5k of the first hook-and-loop fastener 5 is exposed through the first opening portion 15.

The second opening portion 18 is correspondingly provided with the second hook-and-loop fastener 8. The second hook-and-loop fastener 8 is welded to the outer cloth 11 in a state where the second opening portion 18 is closed from the storage space SP side. In the present specification, a region of the outer cloth 11 to which the second hook-and-loop fastener 8 is welded is indicated by a welded region Wpf2. In the example of FIGS. 1 and 2, the welded region Wpf2 has a substantially rectangular frame shape.

A hook surface 8k is formed on the surface of the second hook-and-loop fastener 8, as a fastener surface engageable with the raised surface 5k. The second hook-and-loop fastener 8 is welded to the outer cloth 11 from the inner side of the band 10 in a state where the hook surface 8k of the second hook-and-loop fastener 8 is exposed through the second opening portion 18. The area of the raised surface 5k exposed through the first opening portion 15 of the first hook-and-loop fastener 5 is larger than the area of the hook surface 8k exposed through the second opening portion 18 of the second hook-and-loop fastener 8.

As described above, the first hook-and-loop fastener 5 and the second hook-and-loop fastener 8 are provided such that the raised surface 5k and the hook surface 8k are exposed to the outer surface side of the band 10. When the band 10 is folded back on the end edge ut side and wound around the upper arm part M by using the ring-like member 20, the first hook-and-loop fastener 5 and the second hook-and-loop fastener 8 can be engaged with each other.

As illustrated in FIG. 4, the bag sheet member 9 is welded to the surface of the inner cloth 12 on the storage space SP side (the second member 12i described above). The bag sheet member 9 is a sheet-like member made of a highly stretchable material. The material of the bag sheet member 9 is not particularly limited, but a material containing a resin is preferably used; for example, PVC can be more preferably used.

An edge portion of the bag sheet member 9 is welded to the inner surface (the second member 12i) of the inner cloth 12 over the entire circumference, at a predetermined position with respect to the inner cloth 12. As a result, an airtight space is formed by the bag sheet member 9 and the inner cloth 12. This airtight space functions as the air bag 7. In the present specification, a region of the inner cloth 12 to which the bag sheet member 9 is welded is referred to as a welded region Wps. In the example of FIGS. 1 and 2, the welded region Wps has a substantially rectangular frame shape. The welded region Wps constitutes a second welded region. As illustrated in FIGS. 1 and 2, the welded region Wps is formed on the inner side of the welded region Wp of the outer cloth 11 and the inner cloth 12 that form the storage space SP.

The bag sheet member 9 is provided with a tube connecting portion 7b through which air can be supplied to and discharged from the airtight space. The tube connecting portion 7b is a portion to which an air tube at connected to a main body (not illustrated) of a sphygmomanometer is connected.

The bag sheet member 9 is disposed eccentrically to the end edge it side in the storage space SP. In the band 10, the first hook-and-loop fastener 5 is provided on the end edge it side and has a large area. Therefore, as illustrated in FIGS. 1 and 2, the first hook-and-loop fastener 5 has an overlapped region P overlapping with the bag sheet member 9.

As illustrated in FIG. 1, the upper and lower portions (the welded region

Wpf1a and the welded region Wpf1c) in the drawing extending in the longitudinal direction in the welded region Wpf1 (a substantially U-shaped portion in the example of the drawing) to the outer cloth 11 in the overlapped region P of the first hook-and-loop fastener 5 are positioned on the inner side, in the lateral direction, of the upper and lower portions in the drawing extending in the longitudinal direction in the welded region Wps. Furthermore, a portion (welded region Wpf1d) on the left side in the drawing extending in the lateral direction in the welded region Wpf1 to the outer cloth 11 in the overlapped region P of the first hook-and-loop fastener 5 is positioned on the inner side of the welded region Wps. That is, most of the welded region Wpf1 in the overlapped region P is located on the inner side of the welded region Wps and has almost no overlap with the welded region Wps.

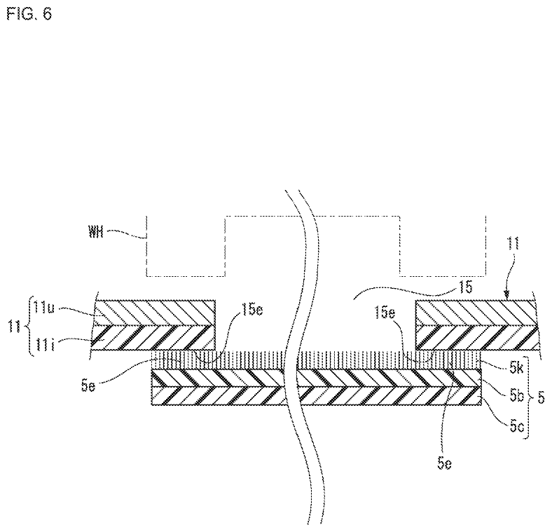

FIG. 6 is an enlarged schematic cross-sectional view illustrating a state before the first hook-and-loop fastener 5 is welded in manufacturing the cuff for measuring blood pressure 1. As illustrated in FIG. 6, in the first hook-and-loop fastener 5, in a state immediately before welding, an edge side portion 5e of the raised surface 5k faces an opening edge portion 15e of the first opening portion 15. In this state, welding is performed by applying a predetermined pressure to the outer cloth 11 by a welding head WH of a welding machine. That is, in a state where the edge side portion 5e of the raised surface 5k and the second member 11i of the opening edge portion 15e are pressed with a predetermined pressure, the second member 11i of the opening edge portion 15e is melted, and the edge side portion 5e of the raised surface 5k enters the melted portion of the second member 11i.

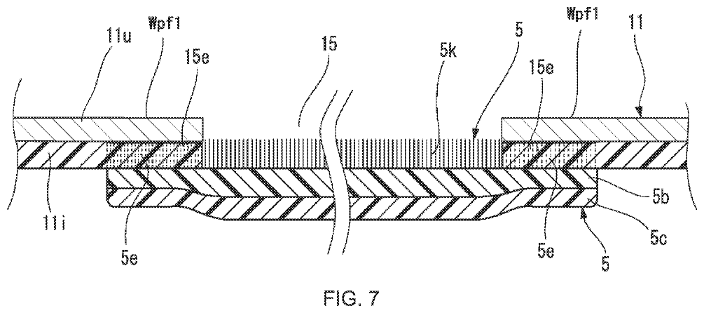

FIG. 7 is an enlarged schematic cross-sectional view illustrating a welded state of the first hook-and-loop fastener 5 illustrated in FIG. 1. As illustrated in FIG. 7, the opening edge portion 15e of the first opening portion 15 and the edge side portion 5e of the raised surface 5k are in a welded state where the raised surface 5k sinks into the opening edge portion 15e. Therefore, in this welding, as can be seen from the structure of the raised surface 5k, the second member 11i is easily engaged with the raised surface 5k, the engagement area can be increased, and the engagement force can be made stronger, as compared with the normal welding between flat surfaces.

As illustrated in FIGS. 6 and 7, the first hook-and-loop fastener 5 has a configuration in which the raised surface 5k is formed on a fastener base 5b and a fastener rear surface member 5c is fixed to a surface of the fastener base 5b opposite to the raised surface 5k with an adhesive or the like. The position of the front edge face of the raised surface 5k on the side opposite to the fastener base 5b side is preferably closer to the outer surface side of the outer cloth 11 than the half-thickness position of the first member 11u of the outer cloth 11 is, and more preferably at the same position as the outer surface of the outer cloth 11. In this manner, the first hook-and-loop fastener 5 and the second hook-and-loop fastener 8 can be favorably joined to each other. In addition, the appearance quality of the cuff can be improved.

For the fastener rear surface member 5c, a raw material having stretchability equal to or lower than that of the first member 11u is used. For the fastener rear surface member 5c, for example, tarpaulin, polyester cloth, a PVC film, or the like can be used. Selecting the raw material of the fastener rear surface member 5c allows appropriate compensation for changes in the stretchability, strength, or the like of the outer cloth 11 due to the formation of the first opening portion 15 in the outer cloth 11.

Like the first hook-and-loop fastener 5, the second hook-and-loop fastener 8 may include a fastener rear surface member, but desirably has the following structure.

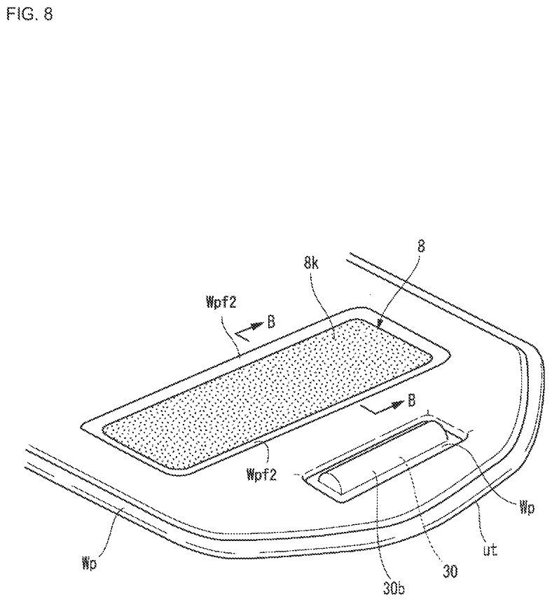

FIG. 8 is an enlarged perspective view illustrating the end edge ut side of the cuff for measuring blood pressure 1 illustrated in FIG. 1. As illustrated in FIG. 8, on the inner side of the welded region Wpf2, the hook surface 8k of the second hook-and-loop fastener 8 is provided at substantially the same height as or slightly lower height than the welded region Wpf2. As will be described later, a stopper portion 30b on the side of the end edge ut of the band 10 is a thick portion necessary for preventing the end edge ut of the band 10 from coming off the ring-like member 20.

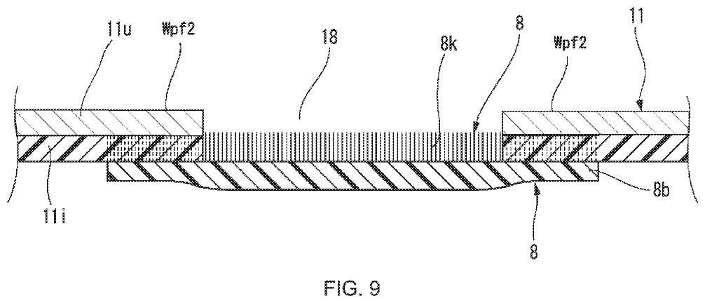

FIG. 9 is a cross-sectional view taken along line B-B of FIG. 8. As illustrated in FIG. 9, unlike the first hook-and-loop fastener 5, the second hook-and-loop fastener 8 has a structure in which a fastener rear surface member is not provided on the side opposite to the hook surface 8k. That is, the second hook-and-loop fastener 8 is constituted only by a fastener base member 8b on which the hook surface 8k is formed. In the second hook-and-loop fastener 8, as in the first hook-and-loop fastener 5, the edge side portion of the hook surface 8k of the outer cloth 11 in contact with the second member 11i is welded to the second member 11i and is firmly joined to the outer cloth 11. The position of the front edge face of the hook surface 8k on the side opposite to the fastener base member 8b side is preferably closer to the outer surface side of the outer cloth 11 than the half-thickness position of the first member 11u of the outer cloth 11 is, and more preferably at the same position as the outer surface of the outer cloth 11. In this manner, the first hook-and-loop fastener 5 and the second hook-and-loop fastener 8 can be favorably joined to each other. In addition, the appearance quality of the cuff can be improved.

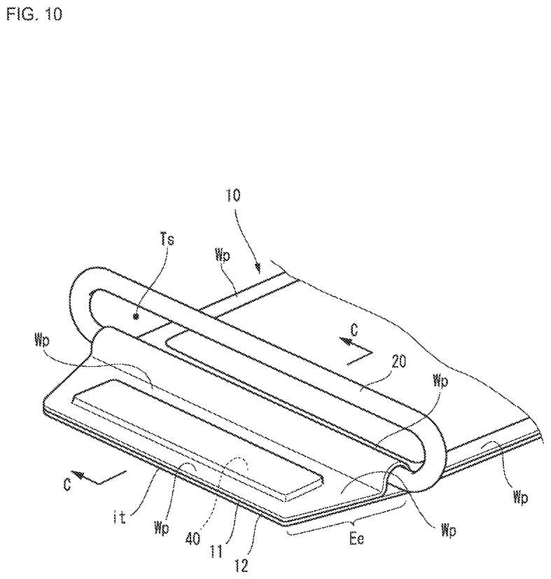

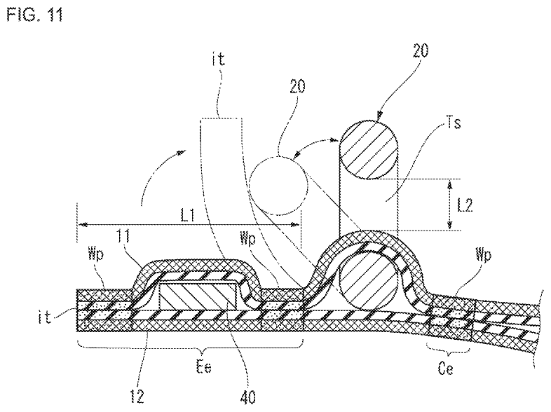

FIG. 10 is an enlarged perspective view illustrating the terminal region Ee of the cuff for measuring blood pressure 1. FIG. 11 is a cross-sectional view of a portion taken along line C-C of FIG. 10. As illustrated in FIG. 10, the ring-like member 20 is held on the band 10 at a position near the end edge it. Specifically, as illustrated in FIG. 11, the ring-like member 20 is attached to the band 10 with the ring-like member 20 sandwiched between the outer cloth 11 and the inner cloth 12. The ring-like member 20 has an oblong shape extending slightly larger than the width of the band 10 in the lateral direction of the band 10.

In the terminal region Ee between the end edge it of the band 10 and the ring-like member 20, there is provided an auxiliary member 40 having a rectangular plate shape in plan view and extending in the lateral direction substantially parallel to the ring-like member 20. The auxiliary member 40 is fixed so as to be sandwiched between the outer cloth 11 and the inner cloth 12. The four peripheral sides of the auxiliary member 40 are surrounded by the welded region Wp, and the auxiliary member 40 is firmly fixed so as not to move in the surrounded space. Here, the welded region Wp around the auxiliary member 40 is formed in the entire region of the terminal region Ee other than a portion where the auxiliary member 40 is disposed.

The region formed by welding the two members has higher rigidity than a configuration in which the two members are fixed by sewing. Although the raw material of the auxiliary member 40 is not particularly limited, the auxiliary member 40 is made of a raw material such as a synthetic resin, which is harder than the outer cloth 11. The entire portion of the terminal region Ee excluding the auxiliary member 40 is the welded region Wp, and further includes the auxiliary member 40, which is hard. Therefore, the terminal region Ee is formed as a region having higher rigidity than the other portion of the band 10.

As illustrated in FIG. 11, in the band 10, the periphery of the attachment portion of the ring-like member 20 is also constituted by the welded region Wp. That is, the outer cloth 11 and the inner cloth 12 are also welded to each other in an adjacent region Ce adjacent to the ring-like member 20 on the opposite side to the terminal region Ee with the ring-like member 20 therebetween.

In this manner, the ring-like member 20 is surrounded by the welded region Wp and is firmly attached so as not to move. The term “not to move” used here means that the ring-like member 20 can be swung around the attachment portion thereof, but the swing axis thereof does not move in the longitudinal direction of the band 10.

As illustrated in FIG. 11, the terminal region Ee is formed such that a width L1 in the longitudinal direction of the band 10 is larger than a width L2 of a gap Ts between the ring-like member 20 located outside the band 10 and the front surface of the band 10. Therefore, it is possible to make the end edge it of the band 10 difficult to enter the gap Ts when the terminal region Ee moves to the ring-like member 20 side or when the ring-like member 20 moves to the terminal region Ee side.

### Manufacturing Process of Cuff for Measuring Blood Pressure 1

Hereinafter, an example of manufacturing the cuff for measuring blood pressure 1 will be described. In this case, the manufacture can be performed by welding components illustrated in FIG. 3 as described below.

The welding step on the outer cloth 11 side will be described. First, the first hook-and-loop fastener 5 is set in a welding machine such that the first opening portion 15 is closed by the first hook-and-loop fastener 5 with the raised surface 5k facing upward and with the outer cloth 11 overlapping with the first hook-and-loop fastener 5. In this state, the second member 11i and the raised surface 5k of the first hook-and-loop fastener 5 in the opening edge portion 15e (see FIG. 6) of the first opening portion 15 are welded to each other. Subsequently, the second hook-and-loop fastener 8 is also welded to the outer cloth 11 like the first hook-and-loop fastener 5.

The welding step on the inner cloth 12 side will be described. First, the tube connecting portion 7b is welded to a predetermined position of the bag sheet member 9. Subsequently, the bag sheet member 9 is welded to the inner surface side of the inner cloth 12. The position of the welded region Wps of the bag sheet member 9 is considered such that the welded region Wps does not overlap with the welded region Wp of the band 10 and most of the welded region Wpf1 of the first hook-and-loop fastener 5. For example, the width dimension of the bag sheet member 9 in the lateral direction of the band 10 is set so as to be positioned on the outer side of the welded region Wpf1 of the first hook-and-loop fastener 5 in the lateral direction. Accordingly, the welded region Wps along the longitudinal direction of the band 10 can be positioned between the welded region Wpf1 of the first hook-and-loop fastener 5 and the welded region Wp of the outer edge of the band 10. As a result, the welded region Wp, the welded region Wps, and the welded region Wpf1, which extend in the longitudinal direction of the band 10, are prevented from overlapping with each other in the thickness direction of the band 10. Since the plurality of welded regions each having rigidity and extending in the longitudinal direction do not overlap with each other, rigidity is prevented from being increased at a specific position of the band 10, and strangeness and discomfort caused by a cured portion when the band 10 is worn are avoided.

Next, the outer cloth 11 and the inner cloth 12 are set in a welding machine such that their inner surface sides face each other. In this state, the outer edges of the outer cloth 11 and the inner cloth 12 are welded. Here, the welding is performed in a region between the adjacent region Ce and the end edge ut.

Next, welding is performed in a portion between the ring-like member 20 and the end edge it (terminal region Ee). The welding is performed with the auxiliary member 40 inserted between the outer cloth 11 and the inner cloth 12. Thereafter, the stopper tube 30 inserted in advance between the outer cloth 11 and the inner cloth 12 is moved to a predetermined position on the end edge ut side, and then the outer periphery of the stopper tube 30 is welded to form the stopper portion 30b. Thus, the manufacturing process of the cuff for measuring blood pressure 1 is completed.

According to the above-described manufacturing process, the cuff for measuring blood pressure 1 has a configuration obtained without sewing in which all the fixed portions are formed of the welded regions. Specifically, in the storage space SP formed between the outer cloth 11 and the inner cloth 12, the first hook-and-loop fastener 5, the second hook-and-loop fastener 8, and the air bag 7 are fixed and stored by welding. Further, the ring-like member 20 and the auxiliary member 40 are attached to the end edge it side of the band 10 by welding. The stopper tube 30 is attached to the end edge ut side of the band 10 by welding. Thus, the entire assembly of the cuff for measuring blood pressure 1 is carried out only by welding.

FIG. 12 is a schematic cross-sectional view illustrating a state where the cuff for measuring blood pressure 1 is being worn on the upper arm part M. As illustrated in FIG. 12, when blood pressure is measured, the cuff for measuring blood pressure 1 is worn on the upper arm part M as the target measurement site. In this case, the end edge ut of the band 10 is passed through the ring-like member 20 in advance. That is, by making the stopper portion 30b pass through the gap Ts of the ring-like member 20, a loop shape of the band 10 is always maintained, and it is easy to wear the band 10 on the upper arm part M.

The arm is passed through the loop of the band 10 formed in this manner such that the air bag 7 is positioned at a predetermined position of the upper arm part M, and the portion of the band 10 through which the ring-like member 20 is passed is pulled so as to be folded back. Then, the band 10 is made to press the upper arm part M with a pressure as appropriate, and the second hook-and-loop fastener 8 is pressed against and fixed to the first hook-and-loop fastener 5. In this winding operation, unexpected bending is not caused to the terminal region Ee due to its rigidity, and the state where the inner surface thereof is in contact with the upper arm part M is maintained (the state illustrated in the drawing). Therefore, in a state where the winding is completed, the ring-like member 20 falls in the folding-back direction, a tip side 20e is always positioned on the outer side of the auxiliary member 40, so that the ring-like member 20 does not come into direct contact with the upper arm part M.

### Effects of Cuff for Measuring Blood Pressure 1

As described above, in the cuff for measuring blood pressure 1, the first hook-and-loop fastener 5 and the second hook-and-loop fastener 8 are provided on the inner side of the band 10. Thus, it is possible to prevent an object from being caught by the first hook-and-loop fastener 5 and the second hook-and-loop fastener 8 when the band 10 is worn, for example. As a result, it is possible to prevent the first hook-and-loop fastener 5 and the second hook-and-loop fastener 8 from being peeled off from each other and improve the durability of the cuff for measuring blood pressure 1.

Further, the first hook-and-loop fastener 5 and the second hook-and-loop fastener 8 are welded to the outer cloth 11. Therefore, the cuff for measuring blood pressure 1 can be easily manufactured and the manufacturing cost can be reduced as compared with the conventional sewing. Furthermore, as compared with a configuration in which the first hook-and-loop fastener 5 and the second hook-and-loop fastener 8 are fixed to the outer surface of the outer cloth 11 by sewing, the appearance quality of the cuff for measuring blood pressure 1 can be improved because the sewn portions and the fastener end edges are not visible.

In addition, in the cuff for measuring blood pressure 1, the first hook-and-loop fastener 5 and the second hook-and-loop fastener 8 can be welded to the outer cloth 11 using a material generally used as the outer cloth 11. Therefore, an existing raw material of the cuff for measuring blood pressure can be used as the band 10 as it is, and an increase in manufacturing cost can be prevented.

In the cuff for measuring blood pressure 1, the first hook-and-loop fastener 5 is provided with the fastener rear surface member 5c having stretchability equal to or lower than that of the first member 11u on the rear surface side of the surface exposed through the first opening portion 15. Accordingly, an increase in stretchability of the outer cloth 11 due to the formation of the first opening portion 15 can be suppressed by the fastener rear surface member 5c. As a result, it is possible to realize the compression state of the target measurement site suitable for blood pressure measurement.

In the cuff for measuring blood pressure 1, the fastener rear surface member 5c is provided on the rear surface side of the first hook-and-loop fastener 5 occupying a large proportion in the outer cloth 11. Therefore, the stretchability of the entire outer cloth 11 can be sufficiently and appropriately adjusted. As a result, it is possible to compress the target measurement site suitably for blood pressure measurement.

In addition, in the cuff for measuring blood pressure 1, the fastener rear surface member 5c is provided on the rear surface side of the first hook-and-loop fastener 5 on the side near the air bag 7 which greatly contributes to the compression of the target measurement site. This allows the stretchability of the outer cloth 11 in the vicinity of the air bag 7 to be adjusted to be suitable therefor. As a result, it is possible to compress the target measurement site suitably for blood pressure measurement.

In addition, in the cuff for measuring blood pressure 1, since the second hook-and-loop fastener 8 is not provided with a fastener rear surface member, it is possible to simplify the configuration and reduce the manufacturing cost without adding more members than necessary. The second hook-and-loop fastener 8 has a small area and is disposed near the end edge ut. Therefore, even when the stretchability of the outer cloth 11 is slightly increased at the portion of the second hook-and-loop fastener 8 of the band 10 due to the presence of the second opening portion 18, the influence on blood pressure measurement can be made slight.

In the cuff for measuring blood pressure 1, the outer cloth 11 and the inner cloth 12 in the terminal region Ee near the ring-like member 20 are welded to each other in a state where the auxiliary member 40 is sandwiched therebetween. Therefore, the rigidity of the terminal region Ee can be enhanced. As a result, it is possible to prevent the terminal region Ee from passing through the ring-like member 20. Therefore, it is possible to prevent a target measurement site from being compressed in a state where the ring-like member 20 is in contact with the target measurement site. Further, the conventional seamed portion at the end edge it of the band 10 can be reduced by welding, which not only improves durability and productivity, but also enhances appearance quality.

In particular, in the cuff for measuring blood pressure 1, since the welded region Wp is formed surrounding the auxiliary member 40 in the terminal region Ee, the rigidity of the terminal region Ee can be effectively increased.

In the cuff for measuring blood pressure 1, the ring-like member 20 is surrounded by the terminal region Ee and the adjacent region Ce and is held in the welded region Wp. Thus, it is possible to attach the ring-like member 20 to the band 10 with a reduced gap for holding the ring-like member 20. Thus, the movable range of the ring-like member 20 can be sufficiently narrowed. As a result, it is possible to more strongly prevent the terminal region Ee from passing through the ring-like member 20.

In the cuff for measuring blood pressure 1, the width L1 of the terminal region Ee is larger than the width L2 of the gap Ts formed by the ring-like member 20. Therefore, it is possible to more strongly prevent the terminal region Ee from passing through the ring-like member 20.

In the cuff for measuring blood pressure 1, the air bag 7 is formed by welding the bag sheet member 9 to the inner cloth 12. Therefore, for example, a step of separately manufacturing the air bag is not necessary, and an operation of incorporating the air bag between the outer cloth 11 and the inner cloth 12 can also be eliminated. As a result, the number of components of the air bag 7 and the number of manufacturing steps of the cuff for measuring blood pressure 1 are reduced, so that the manufacturing cost thereof can be reduced.

In addition, in the cuff for measuring blood pressure 1, since the air bag 7 can be formed by welding to the inner cloth 12, the position where the air bag 7 is disposed can be flexibly determined. For example, as described above, the welded region Wp, the welded region Wps, and the welded region Wpf1, which extend in the longitudinal direction of the band 10, can be designed so as not to overlap with each other in the thickness direction of the band 10. Therefore, it is possible to improve the wearability of the band 10.

In the cuff for measuring blood pressure 1, the bag sheet member 9 with which the air bag 7 is formed is not fixed to the outer cloth 11. Therefore, the outer cloth 11 can be processed freely. As a result, it is possible to increase the degree of freedom in designing the cuff and to reduce the manufacturing cost. In the cuff for measuring blood pressure 1, the bag sheet member 9 is formed as a separate body from the outer cloth 11 and is not fixed to the outer cloth 11. Thus, noise transmitted from the outer cloth 11 side to the air bag 7 can be suppressed, and the accuracy of blood pressure measurement can be improved.

In the cuff for measuring blood pressure 1, the welded region Wpf1d of the welded region Wpf1 between the first hook-and-loop fastener 5 and the outer cloth 11 is located on the inner side of the welded region Wps. Thus, it is possible to prevent hardening of the band 10 in the overlapped region due to the welded region Wps and the welded region Wpf1d overlapping with each other. As a result, it is possible to improve the wearability of the band 10.

### Modified Example of Cuff for Measuring Blood Pressure 1

Although the hook-and-loop fastener is provided on the outer cloth 11 side in the above-described embodiment, the hook-and-loop fastener may be provided on the inner cloth 12 side, depending on how the band 10 is used. That is, the first skin member in the present invention may be either the outer cloth 11 or the inner cloth 12.

In the above-described embodiment, the second hook-and-loop fastener 8 is provided at the end portion on the side opposite to the ring-like member 20 side in the longitudinal direction of the band 10 and has a relatively small area. The first hook-and-loop fastener 5 is provided between the ring-like member 20 and the second hook-and-loop fastener 8 and has a relatively large area. However, the first hook-and-loop fastener in the present invention may be either hook-and-loop fastener regardless of its size.

Further, in the above-described embodiment, both the first hook-and-loop fastener 5 and the second hook-and-loop fastener 8 have been described as hook-and-loop fasteners. However, the first hook-and-loop fastener 5 and the second hook-and-loop fastener 8 only need to be engageable with each other, and one side may be a normal cloth having a simple raised structure. That is, the first member 11u of the outer cloth 11 may be formed of a cloth having a raised structure, and one hook-and-loop fastener engageable with the raised structure of the outer cloth 11 may be welded to the outer cloth 11. Further, in the above-described embodiment, for fastener surfaces, the first hook-and-loop fastener 5 is the raised surface 5k and the second hook-and-loop fastener 8 is the hook surface 8k; however, the present invention is not limited thereto, and a reverse configuration may be adopted.

In the above-described embodiment, the air bag 7 is formed by welding the bag sheet member 9 only to the inner cloth 12 of the outer cloth 11 and the inner cloth 12. However, a reverse configuration may be adopted. That is, the air bag 7 may be formed by welding the bag sheet member 9 only to the outer cloth 11 of the outer cloth 11 and the inner cloth 12. To be specific, the air bag 7 may be formed by welding the bag sheet member 9 to the second member 11i of the outer cloth 11. In this case, the welded region between the outer cloth 11 and the bag sheet member 9 may be provided at the same position as the welded region Wps of FIG. 2 in a plan view. In this configuration, the first hook-and-loop fastener 5 and the welded region Wps of the bag sheet member 9 partly overlap with each other. However, the fastener rear surface member 5c is included in the first hook-and-loop fastener 5. Therefore, in this overlapped portion, the bag sheet member 9 can be welded to the outer cloth 11 with the first hook-and-loop fastener 5 therebetween.

In the above-described embodiment, the ring-like member 20 is attached between the outer cloth 11 and the inner cloth 12, but the present invention is not limited thereto. For example, the ring-like member 20 may be held on the outer surface of the band 10 with another attachment member therebetween.

While various embodiments have been described with reference to the drawings, needless to say, the present invention is not limited to such examples. It will be apparent to those skilled in the art that various changes and modifications can be made within the scope of the claims, and it is understood that these are naturally belong within the technical scope of the present invention. Further, components of the above-described embodiments may be combined as desired within a range that does not depart from the spirit of the present invention.

Note that the present application is based on Japanese Patent Application filed on Jul. 27, 2020 (JP 2020-126734), the content of which is incorporated herein by reference.

### REFERENCE NUMERALS LIST

1 Cuff for measuring blood pressure
5 First hook-and-loop fastener
5c Fastener rear surface member
5e Edge side portion
5k Raised surface (fastener surface)
8k Hook surface (fastener surface)
7 Air bag
7b Tube connecting portion
8 Second hook-and-loop fastener
10 Band
11 Outer cloth (first skin member)
11u, 12u First member
11i, 12i Second member
12 Inner cloth (second skin member)
15 First opening portion
15e Opening edge portion
18 Second opening portion
20 Ring-like member
40 Auxiliary member
Ce Adjacent region
Ee Terminal region
it End edge
ut End edge
Ts Gap
Wp Welded region (first welded region)
Wps Welded region (second welded region)
Wpf1 Welded region (third welded region)
Wpf2 Welded region

1. A cuff for measuring blood pressure including a band enveloping a fluid bag between a first skin member and a second skin member facing the first skin member, an outer surface of the second skin member being to be in contact with a target measurement site, the cuff for measuring blood pressure comprising:
a first opening portion provided in the first skin member; and
a first hook-and-loop fastener welded to the first skin member in a state where the first opening portion is closed from an inner side of the band and a fastener surface is exposed through the first opening portion, wherein
the first skin member includes a layer of a first member having a sheet-like shape and a layer of a second member including a thermoplastic resin and being overlaid on the layer of the first member so as to be on the inner side of the band,
an edge of the first skin member and an edge of the second skin member are welded to each other with the second member between the edge of the first skin member and the edge of the second skin member,
the first skin member and the first hook-and-loop fastener are welded to each other in a state where a hook or a loop at an edge side portion of the fastener surface enters the second member at an edge of the first opening portion of the first skin member, and
a fastener rear surface member having stretchability equal to or lower than stretchability of the first member is fixed to a surface opposite to the fastener surface of the first hook-and-loop fastener.

2. The cuff for measuring blood pressure according to claim 1, further comprising:
a second opening portion provided in the first skin member; and
a second hook-and-loop fastener welded to the first skin member in a state where the second opening portion is closed from the inner side of the band and a fastener surface is exposed through the second opening portion, wherein
the first skin member and the second hook-and-loop fastener are welded to each other in a state where a loop or a hook at an edge side portion of the fastener surface of the second hook-and-loop fastener enters the second member at an edge of the second opening portion of the first skin member,
a fastener rear surface member having stretchability equal to or lower than stretchability of the first member is fixed to a surface opposite to the fastener surface of the second hook-and-loop fastener, and
the first hook-and-loop fastener and the second hook-and-loop fastener are engageable with each other.

3. The cuff for measuring blood pressure according to claim 2, wherein
an area of the fastener surface of the first hook-and-loop fastener exposed through the first opening portion is larger than an area of the fastener surface of the second hook-and-loop fastener exposed through the second opening portion.

4. The cuff for measuring blood pressure according to claim 2, wherein
the first hook-and-loop fastener is disposed closer to the fluid bag than the second hook-and-loop fastener is.
